(19) United States
(12) Patent Application Publication
(10) Pub. No.: US 2019/0262532 A1
(43) Pub. Date: Aug. 29, 2019

(54) SYSTES AND METHODS FOR CONTROLLING BLADDER IRRIGATION

(71) Applicant: SEOUL NATIONAL UNIVERSITY HOSPITAL, Seoul (KR)

(72) Inventors: Seung-June Oh, Seoul (KR); Hyoun-Joong Kong, Seoul (KR)

(73) Assignee: SEOUL NATIONAL UNIVERSITY HOSPITAL, Seoul (KR)

(21) Appl. No.: 16/285,221

(22) Filed: Feb. 26, 2019

(30) Foreign Application Priority Data
Feb. 28, 2018 (KR) 10-2018-0024183

(51) Int. Cl. A61M 3/02

(52) U.S. Cl. CPC A61M 3/0283; A61M 2025/0037; A61M 3/0295

(57) ABSTRACT

The present invention relates to systems and methods for controlling flow through the urinary bladder and, more specifically, systems and methods for controlling continuous bladder irrigation after surgical treatment of lower urinary tract disorders. The control system for continuous bladder irrigation includes a catheter having a first port though which a first fluid enters a bladder and a second port through a second fluid exits the bladder; a first flow speed sensor for measuring a first speed of the first fluid; a second flow speed sensor for measuring a second speed of the second fluid; a control unit for sending, based on the first and second speeds, a warning signal of an abnormality in infusing the first fluid into the bladder and in draining the second fluid from the bladder.

### CROSS-REFERENCE TO PRIOR APPLICATIONS

This application claims priority of Korean Patent Application No. 10-2018-0024183, filed on Feb. 28, 2018, which is all hereby incorporated by reference in its entirety.

### TECHNICAL FIELD

The present invention relates to systems and methods for controlling flow through the urinary bladder and, more specifically, systems and methods for controlling continuous bladder irrigation after surgical treatment for lower urinary tract disorders.

### DESCRIPTION OF THE RELATED ART

In general, to treat lower urinary tract disorders, such as bladder and prostate diseases, a medical professional may insert an indwelling urethral catheter after surgery. These types of surgery are diverse, including open prostatectomy, transurethral resection of bladder tumor, transurethral resection of prostate, or various transurethral laser prostate surgery for benign prostatic hyperplasia, such as potassiumtitanyl-phosphate (KTP) laser prostatectomy, or holmium laser surgery.

After the surgery, the indwelling urethral catheter is inserted into the urethra of the patient and the patient is moved to the recovery room from the operating room with continuous bladder irrigation of sterile fluid. The three-way indwelling urethral catheter has a first passageway through which the sterile fluid flows into the bladder and a second passageway through which the fluid in the bladder exits from the bladder, and the fluid flows continuously through the first and the second passageways to thereby maintain the continuous bladder irrigation.

The reason for maintaining the continuous bladder irrigation using a three-way indwelling urethral catheter is to prevent the formation of blood clots due to bleeding or blood oozing at the surgical bed after the surgery. Partially because of the surgical bed of the transurethral surgery being constantly in contact with fluid such as urine in the bladder and partially because of the nature of the surgical site, it is difficult to perform complete hemostasis such as applying pressure on the site. Even though a surgeon tries his best to perform meticulous coagulation on the surgical bed after resection of the tissue, some degree of oozing causing hematuria from the surgical bed is inevitable. If these blood clots interfere with urinary drainage a urethral catheter, acute problems such as bladder overdistention, urinary retention, or ensuing severe abdominal discomfort may occur. Therefore, continuous fluid irrigation of the surgical bed of the lower urinary tract through an indwelling urethral catheter for certain period of time, i.e. one to several days, is regarded as a routine procedure in most of the transurethral surgery.

The conventional continuous bladder irrigation is performed as follows. A supply of the sterile irrigating fluid is placed at a height of 40-80 cmH₂O and a tube connected to the supply is connected to the first passageway of the indwelling urethral catheter. Then, the sterile irrigating fluid contained in the supply moves along the tube and the first passageway by gravity and then flows into the bladder. The fluid entering the bladder is mixed with blood and urine generated after the surgery and drained through the second passageway of the urethral catheter. Here, the second passageway is connected to a fluid drain tube that is connected to a fluid collector, and the fluid drained through the second passageway moves along the fluid drain tube and is collected in the fluid collector. In general, the degree of hematuria tends to decrease over time after the surgery. But it may vary significantly along the course of the postoperative recovery time not only due to baseline patient characteristics and intraoperative surgical factors, including blood vessel anatomy of the patient, type of surgery, extent of surgical bed, degree of hemostasis, type of energy applied for coagulation, but also due to postoperative patient' physical activity such as leg movement, abdominal pressure, cough and so on. Therefore, frequent adjustment of the speed of inflowing irrigation fluid and corresponding disposal of the collector of drained fluid may be needed over the course of the patient recovery. The continuous bladder irrigation is performed until the degree of bleeding, or hematuria, becomes insignificant so that discontinuation of continuous irrigation will not cause catheter blockage due to blood clot formation. In usual practice, continuous irrigation may be ceased when degree of hematuria in the draining tube become mild. When the hematuria become mild and stable without continuous bladder irrigation for a few hours, the indwelling urethral catheter may be removed from the patient and the patient is discharged from the hospital.

If the color of hematuria in the draining tube is severe, the possibility of formation of blood clots is high, and the inflow rate of the sterile irrigating fluid needs to be increased. To increase the inflow rate, the number of fluid supplies needs to be also increased in proportion to the inflow rate, and accordingly, the number of fluid collectors needs to be increased to handle the increased volume of drained fluid.

However, the conventional continuous bladder irrigation has the following problems. First, monitoring the color of the drained fluid in the draining tube and replacing the fluid supplies and/or the fluid collectors are manually performed by the medical professionals. Considering that there is a considerable number of patients having the transurethral surgery every day, to perform monitoring the color of hematuria and replacing the fluid supplies/collectors requires a considerable amount of manpower. In addition, the tasks may be physically demanding for a female nurse. Considering the weight of the conventional fluid supply/collector and the number of replacements, the task may be a hard work for a female nurse.

Second, the replacement timing of the irrigation fluid supply or the fluid collector cannot be accurately determined. If the inflow fluid supply or the fluid collector is not replaced at the right time, the continuous bladder irrigation may stop, significantly increasing the possibility of blood clot formation.

In addition, the judgment of whether or not the supply of the irrigating fluid into the bladder or the drainage of the fluid from the bladder to the fluid collector is performed smoothly may be manually determined by the medical professionals. If blood clot blocks the catheter due to inappropriate management in the supply/drainage of the irrigating fluid, the medical professionals may immediately need to disconnect the tubing system and then have to irrigate the bladder manually with normal saline commonly using syringe. These events may cause significant inconvenience to both the patient and the medical professional. Moreover, manual irrigation may break the closed system of the irrigation channels, which may cause bacterial contamination of the urinary tract of the patient. As such, there is a need for a system that can automatically monitor and maintain the continuous bladder irrigation.

### Problems Solved

One of the objectives of the present disclosure is to solve the above-mentioned problems of the conventional bladder irrigation systems and to provide systems and methods for continuously monitoring the fluid supply and/or fluid collector so as to accurately inform the medical professionals of the status of the bladder irrigation system and the time to replace the fluid supply and/or fluid collector.

### Solutions to the Problems

In embodiments, a control system for continuous bladder irrigation includes: a three-way urethral catheter including a first port through which a inflow fluid flows into the bladder and a second port through which the fluid in the bladder is drained from the bladder; a flow rate sensor for sensing the speed of the inflow fluid flowing through the first port and/or the second port; and a control unit for outputting a warning signal related to an abnormality in supplying and collecting fluid based on the measured speed of the fluid.

In embodiments, the flow speed sensor may be provided on a first flow passageway along which the inflow fluid flows toward the first port and/or on a second flow passageway along which the fluid drained from the second port flows.

In embodiments, the control system for continuous bladder irrigation may further include: a weight sensor for measuring the weight of the inflow fluid supply for supplying the fluid to the first port or the weight of the fluid collector for collecting the fluid drained through the second port. The control unit may output a warning signal based on the comparison of the measured weights to reference weight values.

In embodiments, the control system for continuous bladder irrigation includes: a three-way urethral catheter including a first port through which a inflow fluid flows into the bladder and a second port through which the fluid flows in the bladder is drained from the bladder a fluid regulator for regulating a flow rate of the inflow fluid flowing through the first port; a pressure sensor for sensing the internal pressure of the bladder; and a control unit for adjusting the fluid regulator or outputting a warning signal related to an abnormality in draining the drained fluid based on the comparison of the measured pressure to a predetermined reference pressure.

In embodiments, the pressure sensor may be provided on the first flow passageway, which is the path along which the inflow fluid flows to the first port and measures the pressure of the fluid.

In embodiments, a control system for continuous bladder irrigation further includes a color sensor for sensing the degree of hematuria of the drained fluid that varies according to the blood cell concentration. The control unit compares the value of the color with a reference value to determine abnormality in the drained fluid.

In embodiments, the control system for continuous bladder irrigation includes: a three-way urethral catheter including a first port through which a fluid flows into the bladder; a plurality of inflow fluid supplies for supplying the fluid to the first port; a plurality of fluid transfer lines that are the passageways of the fluid and connected to the plurality of fluid supplies, respectively; and a valve for providing a three-way fluid transfer path at a point where one fluid transfer line joins another fluid transfer line.

In embodiments, the control unit further includes a control unit for controlling the valve to supply the fluid from one of the plurality of fluid supplies.

In embodiments, a control system for continuous bladder irrigation includes: a three-way urethral catheter including a second port through which a fluid is drained from the bladder; a plurality of fluid collectors in which the drained fluid through the second port is collected; a plurality of drainage fluid transfer lines that are the passageways of the drainage fluids and respectively connected to the plurality of fluid collectors; and a valve for providing a three-way fluid transfer path at a point where one fluid transfer line joins another fluid transfer line.

In embodiments, the control unit further includes a control unit for controlling the valve to collect the drained fluid into one of the plurality of fluid collectors.

### Effects of the Invention

In embodiments, the system may automatically notify the medical professionals if there is a problem in supplying or collecting the fluid, thereby allowing the medical professionals to promptly solve the problem so that the continuous bladder irrigation is successfully performed.

In embodiments, the system may accurately determine the replacement timing of the fluid supply and/or fluid collector, thereby reducing the concern about the additional operation on the patient so that the inconvenience to the patient or medical professionals is significantly reduced.

In embodiments, there is no need to replace the fluid supply and/or the fluid collector individually even when a plurality of fluid supplies or fluid collectors are provided, thereby saving the time and effort of the medical professionals.

### BRIEF DESCRIPTION OF THE DRAWINGS

FIG. 1 shows a schematic diagram of a control system according to the first embodiment of the present disclosure.

FIG. 2 shows a flowchart of an illustrative process performed by the control unit in FIG. 1 according to embodiments of the present disclosure.

FIG. 3 shows a flowchart of an illustrative process performed by the control unit in FIG. 1 according to embodiments of the present disclosure.

FIG. 4 shows a schematic diagram of a control system according to the second embodiment of the present disclosure.

FIG. 5 shows the fluid regulator in FIG. 4 according to embodiments of the present disclosure.

FIG. 6 shows a flowchart of an illustrative process performed by the control unit in FIG. 4 according to embodiments of the present disclosure.

FIG. 7 shows a flowchart of an illustrative process performed by the control unit in FIG. 4 according to embodiments of the present disclosure.

FIG. 8 shows a schematic diagram of a control system according to the third embodiment of the present disclosure.

FIGS. 9A and 9B show flow directions in the first valve in FIG. 8 according to embodiments of the present disclosure.

FIGS. 10A and 10B show flow directions in the second valve in FIG. 8 according to embodiments of the present disclosure.

FIGS. 11A and 11B show flowcharts of illustrative processes performed by the control unit in FIG. 8 according to embodiments of the present disclosure.

FIG. 12 shows an exemplary system that may be used to implement one or more aspects of the present disclosure.

### DETAILED DESCRIPTION OF THE PREFERRED EMBODIMENTS

Preferred embodiments of a control system for controlling continuous bladder irrigation according to the present disclosure will be described in detail with reference to the drawings. In the following description, for purposes of explanation, specific details are set forth in order to provide an understanding of the invention. It will be apparent, however, to one skilled in the art that the invention can be practiced without these details. Furthermore, one skilled in the art will recognize that embodiments of the present invention, described below, may be implemented in a variety of ways, such as a process, an apparatus, a system, a device, or a method on a tangible computer-readable medium. The same reference numerals designate corresponding parts in various figures.

### First Embodiment

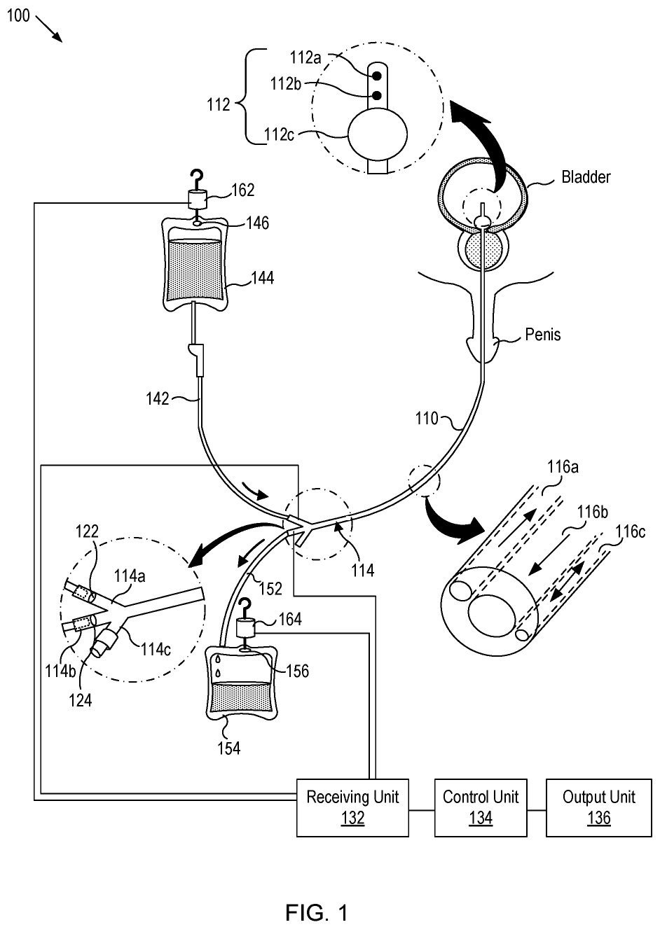

FIG. 1 shows a schematic diagram of a control system according to the first embodiment of the present disclosure. As depicted, the continuous bladder irrigation control system (or shortly control system) 100 includes a three-way urethral catheter 110, flow speed sensors 122 and 124, a receiving unit 132, a control unit 134 and an output unit 136.

In embodiments, the three-way urethral catheter 110 has a total of three lumens or passageways in its catheter design. It includes an inside tip portion 112 and a port portion 114. The inside tip portion 112 is a portion to be inserted into the bladder through the urethra of the patient. The port portion 114 includes an inflow funnel serving for a first port 114a through which a fluid is supplied into the bladder, a drainage funnel serving for a second port 114b through which the fluid in the bladder is drained from the bladder, and a balloon funnel serving for a third port 114c for inflating the balloon 112c at the inside tip of the catheter.

The three-way urethral catheter 110 includes a first passageway 116a, a second passageway 116b, and a third passageway 116c, and the three passageways are physically separated from each other. The first port 114a is in fluid communication with the first passageway 116a for inflow of the irrigating fluid, the second port 114b in fluid communication with the second passageway 116b for drainage of the irrigated fluid, and the third port 114c in fluid communication with the third passageway 116c for balloon. The inside tip portion 112 includes a first communication hole 112a being in fluid communication with the first passage 116a for inflow of the irrigating fluid and a second communication hole 112b being in fluid communication with the second passage 116b for drainage of the irrigated fluid.

When the inside tip portion 112 is inserted into the bladder, the fluid flows in through the third port 114a, irrigating the bladder and then is drained out along the third passageway 116b. The inflated balloon 112c prevents the inside tip portion 112 from being pulled out of the bladder, keeping the urethral catheter in place in a self-retaining manner.

The inflow fluid flowing through the port 114a moves along the first passageway 116a and is infused into the bladder through the first communication hole 112a. The fluid infused into the bladder is mixed with urine and blood oozed out from the surgical bed generated after the surgical procedure, exits through the second communication hole 112b, moves along the second passageway 116b, and flows through the second port 114b.

The first port 114a is connected to the fluid supply 144, which stores the inflow fluid, via the first fluid line 142. The second port 114b is connected to the fluid collector 154 for storing the drained fluid via the second fluid line 152. When the inflow fluid is supplied from the fluid supply 144 to the bladder, a first inflow path of the fluid is formed. Here, the first flow path includes the first fluid line 142 and the first port 114a. When the irrigated fluid is drained from the bladder, a second flow path of the drained fluid is formed. Here, the second flow path includes the second fluid line 152 and the second port 114b.

The flow speed sensors include a first flow speed sensor 122 for measuring the flow speed of the fluid flowing into the bladder and a second flow speed sensor 124 for measuring the flow speed of the drained fluid. The flow speed sensors 122 and 124 may be located on the first and second flow paths, respectively. The flow speed sensors 122 and 124 may be of various types such as electromagnetic type, ultrasonic type, swirl type, thermal type and the like.

When the first flow speed sensor 122 is positioned on the first flow path, the speed of the fluid flowing from the inflow fluid supply 144 to the first port 114a is measured. When the second flow speed sensor 124 is positioned on the second flow path, the speed of the drained fluid moving from the second port 114b to the fluid collector 154 is measured. The flow speed sensors 122 and 124 may measure the speeds simultaneously.

The receiving unit 132 receives the measured flow speeds from the flow speed sensors 122 and 124, and stores the received information in real-time. When the flow speed sensors 122 and 124 are installed in the first flow path and the second flow path, respectively, and send information, the comparison of the speed from the first speed sensor relative to the speed from the second speed sensor is stored in the receiving unit 132.

Based on the information stored in the receiving unit 132, the control unit 134 determines whether there is an abnormality in supplying and draining fluid from the bladder. If there is an abnormality, it is necessary to take proper actions, such as unblock the first fluid line 142 and/or the second fluid line 152, replace the fluid supply 144 if the fluid supply is empty, or replace the fluid collector 154 if the fluid collector is full, so on.

The output unit 136 outputs a warning signal according to the determination of the control unit 134. The warning signal is transmitted to a warning lamp, a warning alarm, etc. installed in a nurse station, and the medical professionals recognize whether there is an abnormality in the system through the warning lamp lighting or the alarm sound.

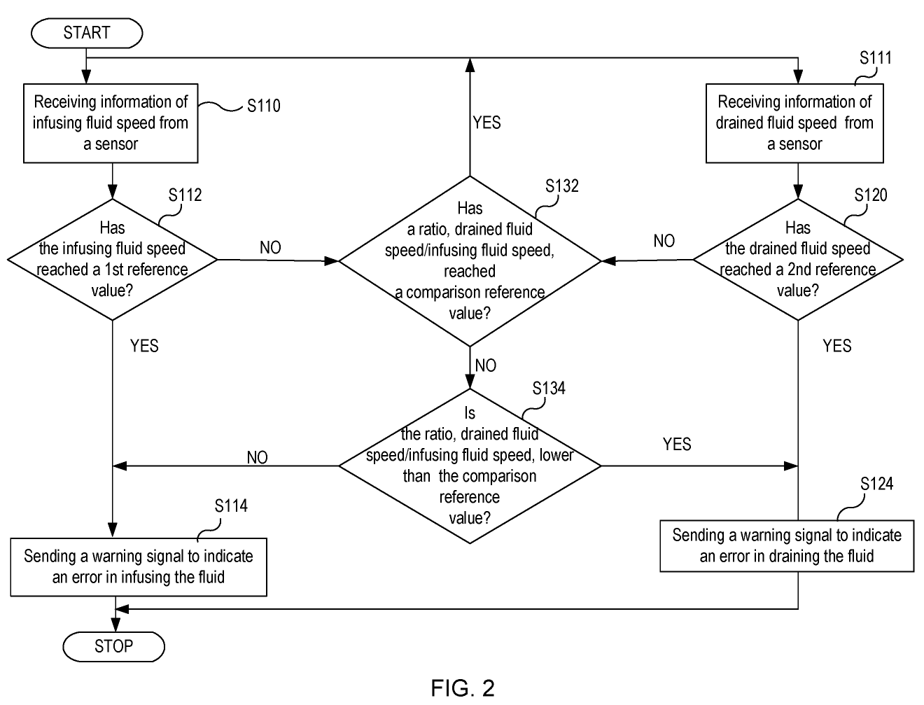

FIG. 2 shows a flowchart of an illustrative process performed by the control unit 134 in FIG. 1 according to embodiments of the present disclosure.

The receiving unit 132 receives and stores the speed information measured by the flow speed sensors 122 and 124 (S110 and S120).

Next, the control unit 134 determines whether the measured speed from the sensor 122 (or 124) has reached a preset first reference value (or a second reference value) based on the measured speed information (S112, S122). Here, the first reference value and/or the second reference value is preset in consideration of various factors such as a patient's physical condition, a patient's posture, and the like.

The control unit 134 determines that there is an abnormality in supplying the fluid when the infusing fluid speed reaches the first reference value and, according to the determination, the output unit 136 outputs a warning signal indicating that there is an abnormality in supplying or draining the fluid (S114). The control unit 134 determines that there is an abnormality in draining the fluid when the drained fluid speed reaches the second reference value and, according to the determination, the output unit 136 outputs a warning signal indicating that there is an abnormality in draining the fluid (S124).

The control unit 134 determined whether the ratio between the drained fluid speed and the infusing fluid speed stored in the receiving unit 132 has reached the preset reference value (i.e., a comparison reference value) (S132).

If the ratio between the drained fluid speed and the infusing fluid speed has reached the comparison reference value, the process proceeds to steps S110 and S120.

If the ratio between the drained fluid speed and the infusing fluid speed has not reached the comparison reference value, the control unit 134 determines whether the ratio is less than the comparison reference value (S134). If the answer to the decision diamond S134 is negative, the process proceeds to step S114. Otherwise, the process proceeds to step S124.

In the first embodiment of the present invention, the control system 100 includes weight sensors 162, 164.

The weight sensors includes a first weight sensor 162 for measuring the weight of the inflow fluid supply 144 and a second weight sensor 164 for measuring the weight of the fluid collector 154. The weight sensors 162, 164 are connected to the suspension holes 146, 156, respectively. However, it should be apparent to those of ordinary skill in the art that the weight sensors 162, 164 may be connected to other suitable locations of the inflow fluid supply 144 and the fluid collector 154.

The receiving unit 132 receives the weight information of the inflow fluid supply 144 and/or the fluid collector 154 measured by the weight sensors 162 and 164, respectively, and stores the received information in real-time.

The control unit 134 determines abnormality in supplying and/or draining the fluid, based on the weight information stored in the receiving unit 132. Here, if there is any abnormality, proper actions may need to be performed, such as unblock the first fluid 142 or second fluid line 152, replace the inflow fluid supply 144 if the inflow fluid supply is empty, or replace the fluid collector 154 if the fluid collector is full.

The output unit 136 sends a warning signal based on the determination of the control unit 134. The warning signal is transmitted to a warning lamp, a warning alarm, etc. installed in the nurse station, and the medical professionals recognize whether there is an abnormality in the patient through the warning lamp lighting or the alarm sound.

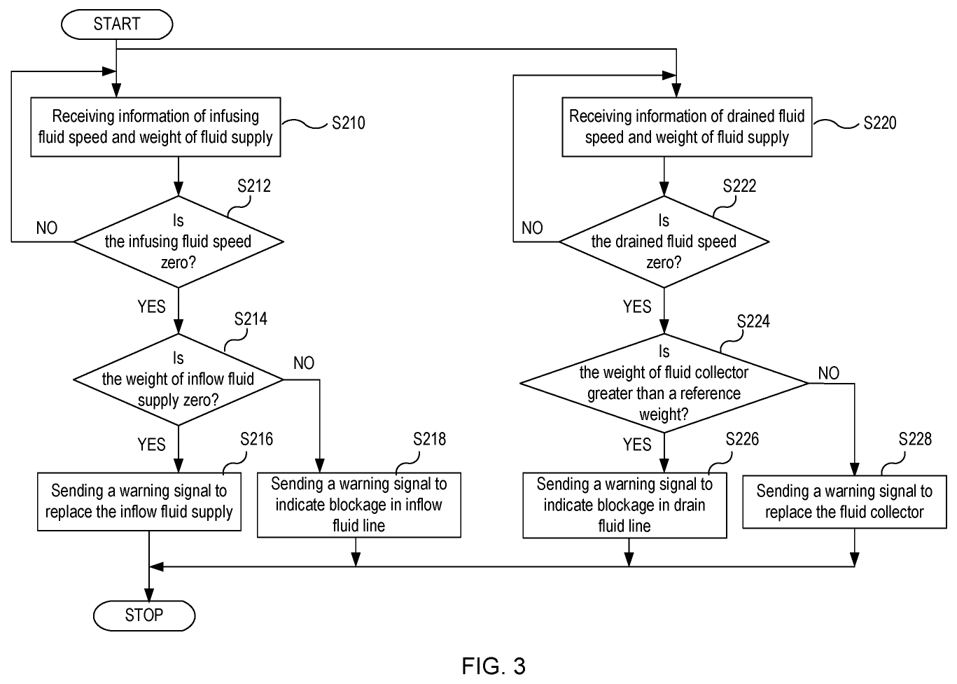

FIG. 3 shows a flowchart of an illustrative process performed by the control unit 134 in FIG. 1 according to embodiments of the present disclosure. As depicted, the control unit 134 controls the abnormity in supplying and draining the fluid.

<Control of Abnormality in Supplying the Inflow Fluid>

The receiving unit 132 receives and stores the infusing fluid speed information from the speed sensor 122 and the weight information from the weight sensor 162 (S210).

The control unit 134 determines whether the infusing fluid speed is zero (S212).

If the infusing fluid speed is not zero, the control unit 134 causes the first flow speed sensor 122 to continuously monitor the inflow fluid speed. Otherwise, the control unit 134 determines whether the weight of the inflow fluid supply 144 is zero, based on the weight information stored in the receiving unit 132 (S214).

If the weight of the inflow fluid supply 144 is zero, the output unit 136 sends a warning signal to replace the inflow fluid supply (S216). Otherwise, the output unit 136 sends a warning signal to unblock the first fluid line 142 (S218).

<Control Abnormality in Draining the Fluid>

The receiving unit 132 receives and stores the drained fluid speed information from the flow speed sensor 124 and the weight information from the weight sensor 164 (S220).

The control unit 134 determines whether the drained fluid speed is zero (S222).

If the drained fluid speed is not zero, the control unit 134 causes the second fluid speed sensor 124 to continuously monitor the fluid speed. Otherwise, the control unit 134 determines whether the weight of the fluid collector 154 is less than the preset weight reference value, based on the weight information stored in the receiving unit 132 (S224).

If the weight of the fluid collector 154 is less than the preset weight reference value, output unit 136 sends a warning sign to unblock the second fluid line 152 (S226). Otherwise, the output unit 136 sends a warning signal to replace the fluid collector (S228).

### Second Embodiment

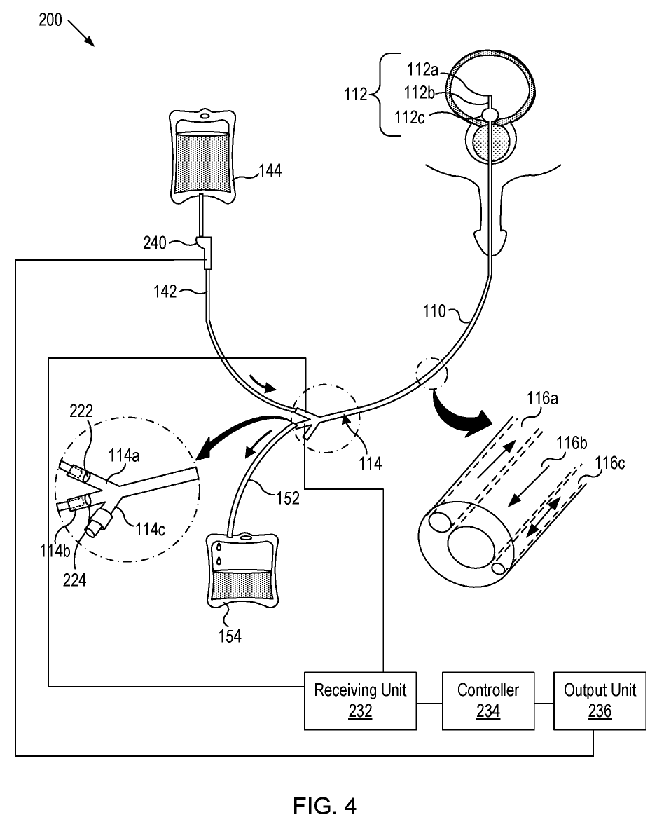

FIG. 4 shows a schematic diagram of a control system 200 according to the second embodiment of the present disclosure. As depicted, the continuous bladder irrigation control system (or shortly control system) 200 includes a catheter 110, a flow regulator 142, a pressure sensor 222, a color sensor 224, a receiving unit 232, a control unit 234 and an output unit 236. In the following sections, the description of the elements common to both the control systems 100 and 200 will not be repeated since the common elements have the similar structure and functions in both systems.

The pressure sensor 222 measures the internal pressure of the bladder and located near the first port 114a of the catheter 110. In alternative embodiments, the pressure sensor 222 may be located in the first fluid line 142 between the fluid supply 144 and the first port 114a. The pressure sensor 222 may be based on (but not limited to) mechanical, electrical, or semiconductor technology.

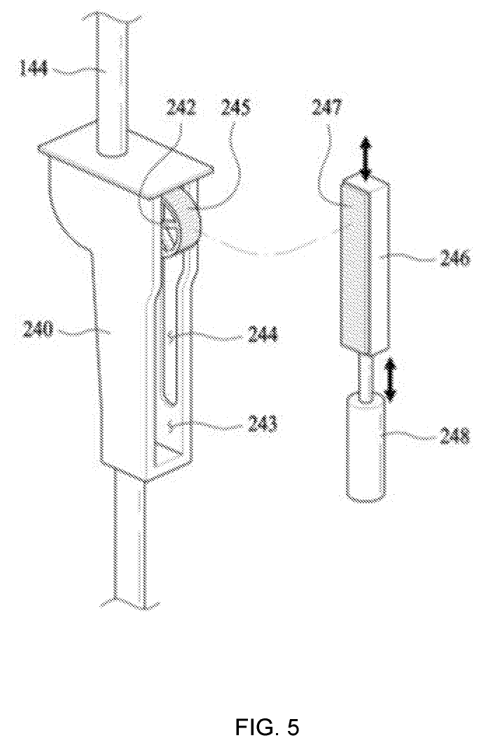

FIG. 5 shows the fluid regulator 142 in FIG. 4 according to embodiments of the present disclosure.

The flow regulator 240 controls the flow rate of fluid that flows from the inflow fluid supply 144 to the first port 114a. The flow regulator 240 may be of any suitable type that is able to control the flow rate and speed, but, for the purpose of illustration, a roller clamp type controller is discussed in the present disclosure.

As depicted in FIG. 5, The flow regulator 142 also includes a hole 243 through which the first fluid line 142 passes and includes a knob 242 that a medical professional manipulates to adjust the cross section area of the low passageway. The knob 242 is guided by a vertical groove 244 and moves along the vertical direction. The depth of the vertical groove 244 increases as the distance from the top increases so that the outer surface of the knob 242 applies more pressure to the first fluid line 142 as the knob 242 moves downwardly and, as a consequence, the flow rate decreases.

A portion of the outer surface of the knob 242 extrudes from the flow regulator 240, and have a first multiple grooves 245 to increase the friction with the finger. At one side of the knob 242 is located an elevating member 246 that contacts the outer surface of the knob 242. A second multiple grooves are formed on one side of the elevating member 246 and engages the first plurality of grooves so that the elevating member 246 moves along the vertical direction as the knob 242 is rotated.

The elevating member 246 is connected to an actuator 248, where the actuator is controlled by an electrical signal to move along with the elevating member 246 in the vertical direction. As discussed below, the output unit 236 sends an electrical signal, either wirelessly or via a wire to move the actuator 248.

The receiving unit 232 receives information of the bladder pressure measured by the pressure sensor 222 and stores the received information in real-time.

The control unit 234 determines whether there is abnormality in supplying the infusing fluid or draining the outflow fluid. For instance, the abnormality in draining fluid includes that the second fluid line 152 is folded or clogged by a substance such as a prostate fragment generated after the surgery.

The output unit 236 outputs an adjustment signal or an abnormality warning signal according to the determination of the control unit 234. The adjustment signal is output to the flow regulator 240 and the warning signal is output to the nurse station where the patient or the medical professional is located.

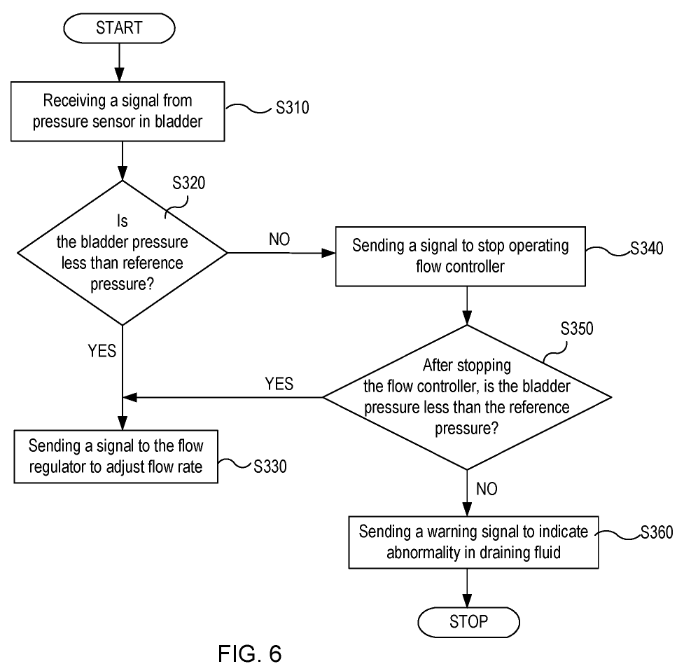

FIG. 6 shows a flowchart of an illustrative process performed by the control unit 234 in FIG. 4 according to embodiments of the present disclosure. The receiving unit 232 receives and stored the information of pressure measured by the pressure sensor 222 (S310).

The control unit 234 determines whether the bladder pressure has reached the preset reference pressure, considering various factors, such as patient's physical condition, posture, etc. (S320).

If the answer to the determination diamond S320 is positive, the output unit 236 sends a signal to the flow regulator 240 to increase or decrease the fluid speed (S330). Otherwise, the control unit 234 determines that the bladder pressure is abnormal and the output unit 236 sends a signal to stop operating the flow regulator 240 (S340).

After outputting a signal for stopping the operation of the fluid regulator 240, the control unit 234 determines whether the bladder pressure is less than the reference pressure (S350).

If the answer to the determination diamond S350 is positive, the process proceeds to step 350. Otherwise, the output unit 236 determines that there is an abnormality in draining the outflow fluid and sends a warning signal (S360).

The continuous bladder irrigation system 200 further includes a color sensor 224 for determining the degree of hematuria.

The color sensor 224 senses a degree of hematuria that is generated when the degree of near-infrared absorbance differs according to the red blood cell concentration included in the drained fluid. According to the second embodiment of the present invention, the color sensor 224 is installed in the second port 114b. In accordance with another embodiment of the present invention, the color sensor 224 is positioned on the second flow passageway, through which the drained fluid travels from the second port 114b along the second fluid line 152 to the fluid collector 154.

The receiving unit 232 receives the information of color that is measured by the color sensor 224 and stores the information in real-time.

The control unit 234 determines abnormality in supplying the infusing fluid or draining the outflow fluid based on the information of color stored in the receiving unit 232.

The output unit 236 sends a warning signal according to the determination of the control unit 234. The warning signal is transmitted to a warning lamp, a warning alarm, etc. installed in the nurse station, and the medical professionals recognize whether there is an abnormality in the system through the warning lamp lighting or the alarm sound.

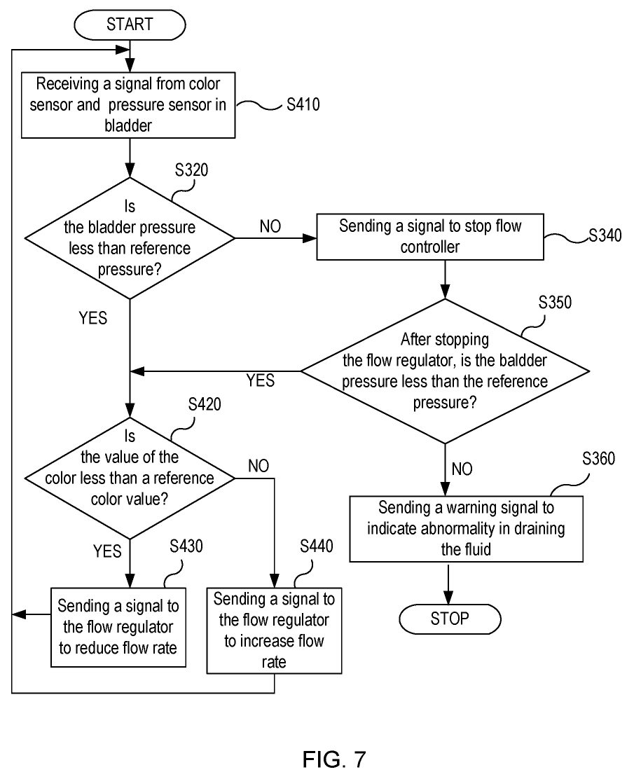

FIG. 7 shows a flowchart of an illustrative process performed by the control unit 234 in FIG. 4 according to embodiments of the present disclosure. In the following section, the control steps of the control unit 234 is described in detail. However, the control steps of the control unit 234 based on the information from the pressure sensor 222 and the color sensor 224 are similar to the steps of the control unit 234 based on the information from the pressure sensor 222, as described above. As such, description of the same steps is not repeated.

The receiving unit 232 receives the information of the pressure measured by the pressure sensor 222 and the information of the color measure by the color sensor 224, and stores the received information (S410).

At step S320, the control unit 234 determines whether the bladder pressure is less than the reference pressure. If the answer to the decision diamond S320 is positive, the control unit 234 determines whether the value of the color received from the color sensor 224 is less than the reference color value (S420). The reference color value may be determined considering various factors, such as the speed of infusing fluid, the patient's condition after the operation, etc. In embodiments, the nurse or medical professionals may set the reference color value at the time when the irrigation system is set up for the patient.

If the answer to the decision diamond S420 is positive, the control unit 234 concludes that the blood cell concentration in the drained fluid is low and the output unit 235 sends a control signal to the flow regulator 240 to reduce the speed of the infusing fluid (S430), and the control unit 234 determines whether the bladder pressure is less than the reference pressure. If the answer to the decision diamond S420 is negative, the control unit 234 concludes that blood cell concentration in the drained fluid is high and the output unit 235 sends a control signal to the flow regulator 240 to increase the speed of the infusing fluid (S440) and the control unit 234 determines whether the bladder pressure is less than the reference pressure.

In embodiments, the output unit 236 may concludes that the number of blood cells in the drained fluid is low and sends a signal to stop operating the flow regulator 240 if the value of the color measured by the color sensor 224 is below a reference value.

In embodiments, the sensors 222 and 224, flow regulator 240, receiving unit 232, controller 234, and output unit 236 may form a feedback system for controlling the system 200. It is noted that the sensors and flow regulator may be electrically coupled to the receiving unit and output unit through wires or wireless communications channels.

### Third Embodiment

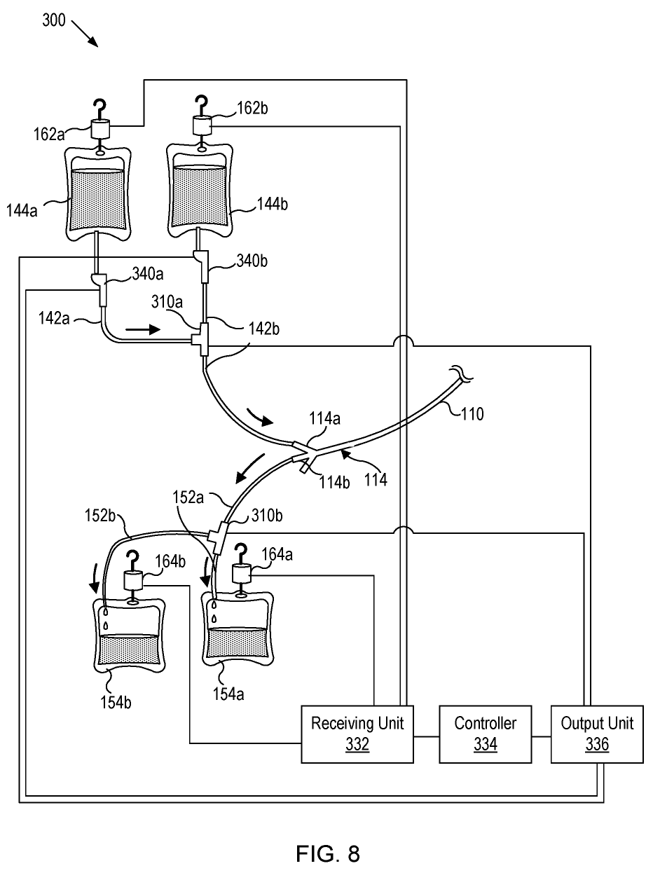

FIG. 8 shows a schematic diagram of a control system (or shortly control system) 300 according to a third embodiment of the present disclosure. As depicted, the control system 300 includes valves 310a, 310b, a receiving unit 332, a control unit 334, and an output unit 336. In the following sections, the description of the elements common to both the control systems 100 and 300 will not be repeated since the common elements have the similar structure and functions in both systems.

The control system 300 includes a plurality of inflow fluid supplies (or, shortly fluid supplies) 144a, 144b and a plurality of fluid collectors 154a, 154b. The control system 300 further includes a plurality of weight sensors 162a, 162b for measuring the weights of the plurality of fluid supplies 144a, 144b, respectively, and a plurality of weight sensors 164a, 164b for measuring the weights of the plurality of fluid collectors 154a, 154b, respectively.

The plurality of fluid supplies 144a, 144b are connected to a plurality of inflow fluid lines 142a, 142b, respectively. As depicted in FIG. 8, the first fluid line 142a, which is connected to the first fluid supply 144a, is connected to the second fluid line 142b, and both the first and second fluid lines are connected to the first port 114a. It should be apparent to those of ordinary skill in the art that the control system 300 may include more than two fluid supplies and two fluid lines.

Similarly, the plurality of fluid collectors 154a, 154b are connected to a plurality of fluid lines 152a, 152b, respectively. As depicted in FIG. 8, the first drained fluid line 152a, which is connected to the first fluid collector 154a, is connected to the second drained fluid line 152b, and both the first and second drained fluid lines are connected to the second port 114b. It should be apparent to those of ordinary skill in the art that the control system 300 may include more than two drained fluid collectors and two drained fluid line.

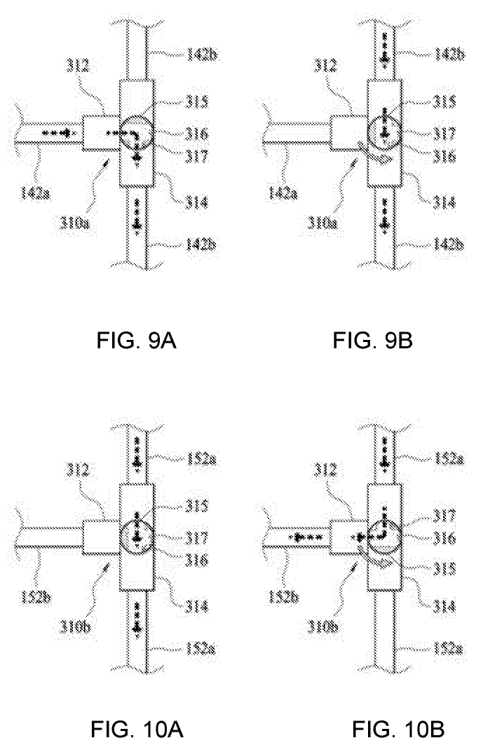

FIGS. 9A and 9B show flow directions in a first valve in FIG. 8 according to embodiments of the present disclosure. FIGS. 10A and 10B show flow directions in a second valve in FIG. 8 according to embodiments of the present disclosure.

The valves 310a, 310b are disposed at the junctions where the fluid lines are connected. As depicted in FIGS. 9A-10B, the valve 310a (or 310b) includes a horizontal section 312 and a vertical section 314 that are configured in a “T” shape, and a rotating member 315 is disposed at the location where the horizontal section meets the vertical section.

As depicted in FIGS. 9A-10B, the horizontal section 312 of the valve 310a (or 310b) is connected to the first infusing fluid line 142a (or the second drained fluid line 152b). Similarly, the vertical section 314 of the valve 310a (or 301b) is connected to the second infusing fluid line 142b (or the first drained fluid line 152a). It is noted that the number of valves may be changed depending on the number of fluid supplies and/or fluid collectors.

In the rotating member 315, a first control hole 316 and a second control hole 317 are formed, where the first control hole 316 extends between two ends and the second control hole 317 extends from one end to a center.

Depending on the rotation of the rotating member 315, the flow direction in the valves 315 changes. When the valve 310a is configured as shown in FIG. 9A, the infusing fluid flows from the first infusing fluid line 142a to the second infusing fluid line 142b through the control hole 316 and the second control hole 317. When the valve 310a is configured as shown in FIG. 9B, the fluid flows downwardly through the second infusing fluid line 142b and the control hole 316.

When the valve 310b is configured as shown in FIG. 10A, the drained fluid flows downwardly through the second drained fluid line 152a and the control hole 316. When the valve 310b is configured as shown in FIG. 10B, the drained fluid flows from the first drained fluid line 152a to the second drained fluid line 152b through the control hole 316 and the second control hole 317.

In embodiments, each valve may include a motor that is mechanically coupled to the rotating member 315 and provides a rotational force to the rotating member 315 so that the rotating member is able to rotate, as shown in FIGS. 9A-10B. The output unit 336 may send an electrical signal to the motor wirelessly (or through a wire) to control the rotating member 315.

The receiving unit 332 receives the weight information from the weight sensors 162a, 162b, 164a, 164b and stores the information in real-time.

The control unit 334 determines whether the weight of the fluid supply's 144a, 144b (or the fluid collectors 154a, 154b) is zero, based on the stored information.

The output unit 336 sends a signal for controlling the valves 310a, 310b and a signal for replacing the inflow fluid supplies 144a, 144b and/or fluid collectors 154a, 154b. The signal for the replacement is an electrical light signal or an audible signal for the medical professionals.

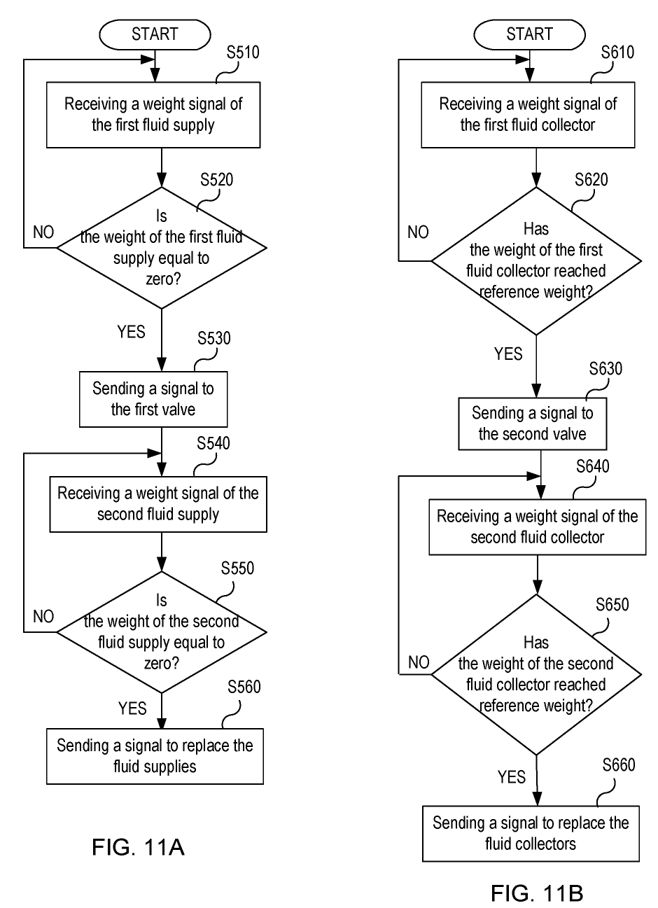

FIGS. 11A and 11B show flowcharts of illustrative processes performed by the control system 300 in FIG. 8 according to embodiments of the present disclosure. For the purpose of illustration, the control system 300 is assumed to have two fluid supply 144a, 144b and two fluid collectors 154a, 154b.

<In a Case where Two Fluid Supplies are Used>

The receiving unit 332 receives the weight information of the first fluid supply 144a from a weight sensor 162a and stores the information (S510). Then, the control unit 336 determines whether the weight of the first fluid supply 144a is equal to zero (S520). If the weight is not equal to zero, the control unit 334 determines that the first fluid supply 144a is not empty and controls the first valve unit 310a to be in the position shown in FIG. 9A. More specifically, the control unit 334 causes the output unit 336 to send a signal to the motor connected to the rotating member 315 (S530) so that the inflow fluid from the first fluid supply 144a flows into the first port 114a. Then, the process proceeds to step S510 so that the control unit 334 can check if the weight of the first fluid supply 144a is equal to zero.

If the answer to the decision diamond S520 is positive, the control unit 334 determines that the first fluid supply 144a does not contain any fluid and causes the output unit 336 sends a signal to the motor so that the first valve 310a is in the position of FIG. 9B and the second fluid supply starts providing the fluid (S530).

The receiving unit 332 receives the weight information of the second fluid supply 144b from the second weight sensor 162b and stores the information (S540).

Then the control unit 334 determines whether the weight of the second fluid supply 144b is equal to zero (S530). If the weight is not equal to zero, the control unit 334 determines that the second fluid supply 144b is not empty and repeat step S540. If the weight is equal to zero, the control unit 334 determines that the second fluid supply 144b is empty and sends a warning signal that the fluid supplies need to be replaced (S560). At step S560, the control unit 334 determines that the weights measured by the two weight sensors 162a, 162b are both equal to zero.

<In Case where Two Fluid Collectors are Used>

The receiving unit 332 receives the information of the first fluid collector 154a from the first weight sensor 164a and stores the information (S610). The control unit 334 controls the second valve 310b so the second valve 310b is in the position shown in FIG. 10A. More specifically, the control unit 334 causes the output unit 336 sends a control signal to the rotating member 315. Then, the drained fluid from the second port 114b is collected by the first fluid collector 154a.

The control unit 334 determines if the weight of the first fluid collector 154a has reached a preset reference weight (S620). If weight has not reached the preset reference weight, the control unit 334 determines that there is no need to replace the first fluid collector 154a and repeat step S610. If the answer to the decision diamond S620 is positive, the control unit 334 determines that the first fluid collector 154a needs to be replaced and causes the output unit 336 to send a signal to the second valve 310b so that the second valve is in the position shown in FIG. 10B (S630). Then, the drained fluid is collected by the second fluid collector 154b.

Next, the receiving unit 332 receives the weight information of the second fluid collector 154b from the second weight sensor 164b and stores the information (S640).

Then, the control unit 334 determines whether the weight of the second fluid collector 154b has reached a preset reference weight (S650). If the answer to the decision diamond S650 is negative, the control unit 334 determines that it is not necessary to replace the second fluid collector 154b and repeat the step S640. Otherwise, the control unit 334 determines that the second fluid collector 154b is not able to collect the drained fluid any more, and causes the output unit 336 to send a warning signal that the fluid collectors 154a, 154b need to be replaced. At step S660, the control unit 334 determines that the weights measured by the two weight sensors 164a, 164b indicate that the fluid collectors 154a, 154b cannot collect the drained fluid any more.

The sensors 162a, 162b, 164a, and 164b, valves 310a, 301b, flow regulators 340a, 340b, receiving unit 332, controller 334, and output unit 336 may form a feedback system for controlling the system 300. It is noted that the sensors, valves and flow regulators may be electrically coupled to the receiving unit and output unit through wires or wireless communication channels.

In embodiments, in the control systems 100, 200, and 300, the signals from the output units 136, 236, 336 may be transmitted to the same output device or to different output devices. For instance, the warning signal indicating blockage of the infusing fluid line 142 and the warning signal indicating replacement of the fluid supply 144 may be sent to different output devices.

In embodiments, one or more computing system may be configured to perform one or more of the methods, functions, and/or operations presented herein. Systems that implement at least one or more of the methods, functions, and/or operations described herein may have an application or applications operating on at least one computing system. The computing system may have one or more computers and one or more databases. The computer system may be a single system, a distributed system, a cloud-based computer system, or a combination thereof.

It shall be noted that the present disclosure may be implemented in any instruction-execution/computing device or system capable of processing data, including, without limitation phones, laptop computers, desktop computers, and servers. The present disclosure may also be implemented into other computing devices and systems. Furthermore, aspects of the present disclosure may be implemented in a wide variety of ways including software (including firmware), hardware, or combinations thereof. For example, the functions to practice various aspects of the present disclosure may be performed by components that are implemented in a wide variety of ways including discrete logic components, one or more application specific integrated circuits (ASICs), and/or program-controlled processors. It shall be noted that the manner in which these items are implemented is not critical to the present disclosure.

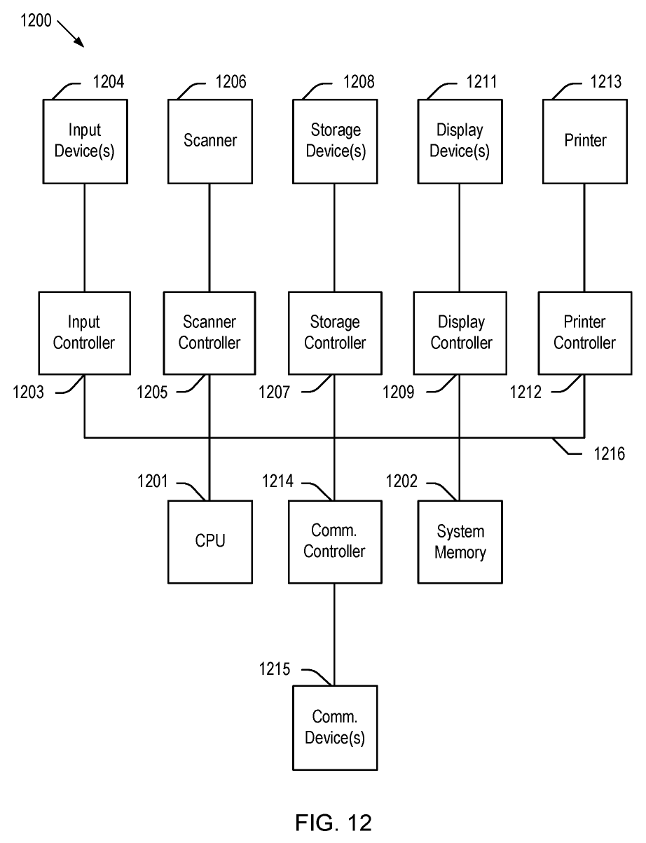

Having described the details of the disclosure, an exemplary system 1200, which may be used to implement one or more aspects of the present disclosure, will now be described with reference to FIG. 12. Each device (such as receiving unit, control unit, output unit) in FIGS. 1-11B may include one or more components in the system 1200. As illustrated in FIG. 12, system 1200 includes a central processing unit (CPU) 1201 that provides computing resources and controls the computer. CPU 1201 may be implemented with a microprocessor or the like, and may also include a graphics processor and/or a floating point coprocessor for mathematical computations. System 1200 may also include a system memory 1202, which may be in the form of random-access memory (RAM) and read-only memory (ROM).

A number of controllers and peripheral devices may also be provided, as shown in FIG. 12. An input controller 1203 represents an interface to various input device(s) 1204, such as a keyboard, mouse, or stylus. There may also be a scanner controller 1205, which communicates with a scanner 1206. System 1200 may also include a storage controller 1207 for interfacing with one or more storage devices 1208 each of which includes a storage medium such as magnetic tape or disk, or an optical medium that might be used to record programs of instructions for operating systems, utilities and applications which may include embodiments of programs that implement various aspects of the present disclosure. Storage device(s) 1208 may also be used to store processed data or data to be processed in accordance with the present disclosure. System 1200 may also include a display controller 1209 for providing an interface to a display device 1211, which may be a cathode ray tube (CRT), a thin film transistor (TFT) display, or other type of display. System 1200 may also include a printer controller 1212 for communicating with a printer 1213. A communications controller 1214 may interface with one or more communication devices 1215, which enables system 1200 to connect to remote devices through any of a variety of networks including the Internet, an Ethernet cloud, an FCoE/DCB cloud, a local area network (LAN), a wide area network (WAN), a storage area network (SAN) or through any suitable electromagnetic carrier signals including infrared signals.

In the illustrated system, all major system components may connect to a bus 1216, which may represent more than one physical bus. However, various system components may or may not be in physical proximity to one another. For example, input data and/or output data may be remotely transmitted from one physical location to another. In addition, programs that implement various aspects of this disclosure may be accessed from a remote location (e.g., a server) over a network. Such data and/or programs may be conveyed through any of a variety of machine-readable medium including, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices.

Embodiments of the present disclosure may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present disclosure. One skilled in the art will also recognize that a number of the elements described above may be physically and/or functionally separated into sub-modules or combined together.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

[Reference numerals]

100: continuous bladder irrigation control system according to the first
embodiment

| | |
|---|---|
| 110: three-way urethral catheter | 112: inside tip portion |
| 112a: first communication hole | 112b: second communication hole |
| 112c: inflating balloon portion | 114: port portion |
| 114a: first port | 114b: second port |
| 114c: third port | 116a: first passageway |
| 116b: second passageway | 116c: third passageway |
| 122: first flow speed sensor | 124: second flow speed sensor |
| 130: control unit | 142: first fluid line |
| 144: inflow fluid supply | 152: second fluid line |
| 154: fluid collector | 162: first weight sensor |
| 164: second weight sensor | |

200: continuous bladder irrigation control system according to the second
embodiment

| | |
|---|---|
| 222: pressure sensor | 224: color sensor |
| 230: control unit | 240: flow regulator |
| 242: knob | 243: hole |
| 244: vertical groove | 245: multiple grooves |
| 246: elevating member | 247: multiple grooves |
| 248: actuator | |

300: continuous bladder irrigation control system according to the third
embodiment

| | |
|---|---|
| 310: valve | 312: horizontal section |
| 314: vertical section | 315: rotating member |
| 316: first control hall | 317: second control ball |
| 330: control unit | 340: flow regulator |

What is claimed is:

1. A control system for continuous bladder irrigation, comprising:
a urethral catheter having a first port though which a first fluid enters a bladder and a second port through a second fluid exits the bladder;
a first flow speed sensor for measuring a first speed of the first fluid;
a second flow speed sensor for measuring a second speed of the second fluid;
a control unit for sending, based on the first and second speeds, a warning signal of an abnormality in infusing the first fluid into the bladder and in draining the second fluid from the bladder.

2. The control system of claim 1, wherein the first flow speed sensor is disposed in a first passageway through which the first fluid flows to the first port and wherein the second flow speed sensor is disposed in a second passageway through which the second fluid flows from the second port.

3. The control system of claim 1, wherein the control unit sends the warning signal based on a comparison between the first speed and the second speed.

4. The control system of claim 1, further comprising:
a first weight sensor for measuring a first weight of an inflow fluid supply for providing the first fluid; and
a second weight sensor for measuring a second weight of a fluid collector for collecting the second fluid,
wherein the control unit sends a warning signal, based on a comparison of the first and second weights to a reference weight.

5. A control system for continuous bladder irrigation, comprising:
a urethral catheter having a first port though which a first fluid enters a bladder and a second port through a second fluid exits the bladder;
a flow regulator for controlling a speed of the first fluid;
a pressure sensor for measuring an internal pressure of the bladder; and
a control unit for sending, based on a comparison between the internal pressure and a reference pressure, a control signal for controlling the flow regulator and sending a warning signal of an abnormality in draining the second fluid from the bladder.

6. The control system of claim 5, wherein the pressure sensor is disposed in a first flow passageway through which the first fluid flows to the first port.

7. The control system of claim 5, further comprising:
a color sensor for measuring the degree of hematuria of the second fluid, the color changes according to a concentration of blood cells in the second fluid;
wherein the control unit determines an abnormality in draining the second fluid from the bladder based on a comparison of the color to a reference color.

8. A control system for continuous bladder irrigation, comprising:
a catheter having a first port though which a inflow fluid enters a bladder;
a plurality of inflow fluid supplies for providing the fluid to the first port;
a plurality of inflow fluid lines connected to the plurality of fluid supplies, respectively; and
a valve providing a three-way fluid passageway at a point where one of the plurality of fluid lines joins an other one of the plurality of fluid lines.

9. The control system of claim 8, further comprising:
a control unit for controlling the valve so that only one of the plurality of fluid supplies the fluid to the first port.

10. A control system for continuous bladder irrigation, comprising:
a catheter having a second port though which a fluid exits a bladder;
a plurality of fluid collectors for collecting the fluid from the first port;
a plurality of fluid lines connected to the plurality of fluid collectors, respectively; and
a valve providing a three-way fluid passageway at a point where one of the plurality of fluid lines joins an other one of the plurality of fluid lines.

11. The control system of claim 10, further comprising:
a control unit for controlling the valve so that only one of the plurality of fluid collectors collects the fluid from the second port.
